(19) United States
(12) Patent Application Publication
(10) Pub. No.: US 2009/0182003 A1
(43) Pub. Date: Jul. 16, 2009

(54) Methods and Compositions for Treatment Cell Proliferative Disorders

(75) Inventors: David A. Sinclair, Chestnut Hill, MA (US); Joseph A. Baur, Merion Station, PA (US); Sean M. Armour, Roxbury Crossing, MA (US)

Correspondence Address: Harvard University & Medical School;c/o Wolf, Greenfield & Sacks, P.C., 600 Atlantic Avenue, Boston, MA, 02210-2206, US

(73) Assignee: President and Fellows of Harvard College, Cambridge, MA (US)

(21) Appl. No.: 12/087,631

(22) PCT Filed: Jan. 12, 2007
(86) PCT No.: PCT/US2007/060504

§ 371 Date: Dec. 17, 2008

Related U.S. Application Data
(60) Provisional application No. 60/759,124, filed on Jan. 13, 2006.

(51) Int. Cl. A61K 31/436; A61K 31/05; A61P 35/00; A61P 35/02

(52) U.S. Cl. 514/291; 514/733

(57) ABSTRACT

The present invention provides methods for treating and preventing cell proliferative disorders and conditions comprising administering to a subject resveratrol or an analog thereof.

### RELATED APPLICATIONS

This application claims the benefit of priority to U.S. provisional application 60/759,124, filed Jan. 13, 2006, which is incorporated by reference in its entirety.

### GOVERNMENT SUPPORT

This invention was made with government support under Grant number R01 AG19972 awarded by the National Institutes of Health. The government has certain rights in this invention.

### BACKGROUND

Protein kinases are a family of enzymes that catalyze phosphorylation of the hydroxyl group of specific tyrosine, serine, or threonine residues in proteins. Typically, such phosphorylation dramatically perturbs the function of the protein, and thus protein kinases are pivotal in the regulation of a wide variety of cellular processes, including metabolism, cell proliferation, cell differentiation, and cell survival. Of the many different cellular functions in which the activity of protein kinases is known to be required, some processes represent attractive targets for therapeutic intervention for certain disease states. Two examples are angiogenesis and cell-cycle control, in which protein kinases play a pivotal role; these processes are essential for the growth of solid tumors as well as for other diseases.

Protein kinases play a crucial role in cell-cycle control. Uncontrolled cell proliferation is the insignia of cancer. Cell proliferation in response to various stimuli is manifested by a de-regulation of the cell division cycle, the process by which cells multiply and divide. Tumor cells typically have damage to the genes that directly or indirectly regulate progression through the cell division cycle. Agents that reduce and/or prevent uncontrolled cell death are needed for treating cancer and other disorders characterized by uncontrolled cell proliferation Further, there remains an unmet need for treating and preventing disorders that are characterized by uncontrolled cell proliferation.

### SUMMARY

Described herein are methods for treating and preventing cell proliferative disorders, e.g., cancer, and conditions in which it is desirable to reduce cell proliferation. A method may comprise administering to a subject resveratrol or an analog thereof.

### BRIEF DESCRIPTION OF THE DRAWINGS

FIG. 1 shows graphs of the activity (as a percentage of the control) of human JAK2 in the presence of 10 and 100 μM ATP (panels A and B, respectively) and various concentrations of resveratrol.

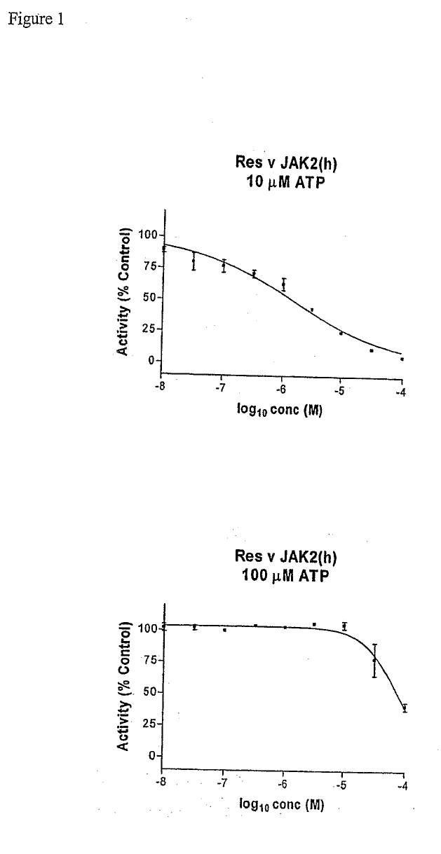

FIG. 2 shows graphs of the activity (as a percentage of the control) of human pim-1 in the presence of 10 or 100 μM ATP (panels A and B, respectively) and various concentrations of resveratrol.

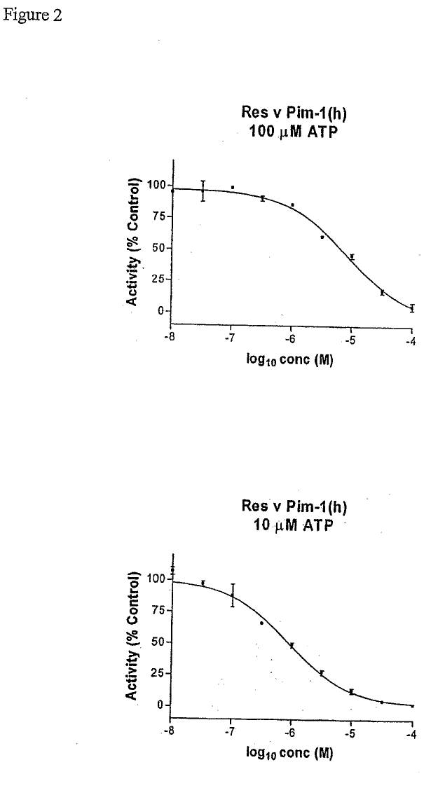

FIG. 3 shows graphs of the activity (as a percentage of the control) of human pim-2 (panel A) and a control enzyme (Auroroa-A) (panel B) in the presence of 10 μM ATP and various concentrations of resveratrol-4-glucoronide, a metabolite of resveratrol.

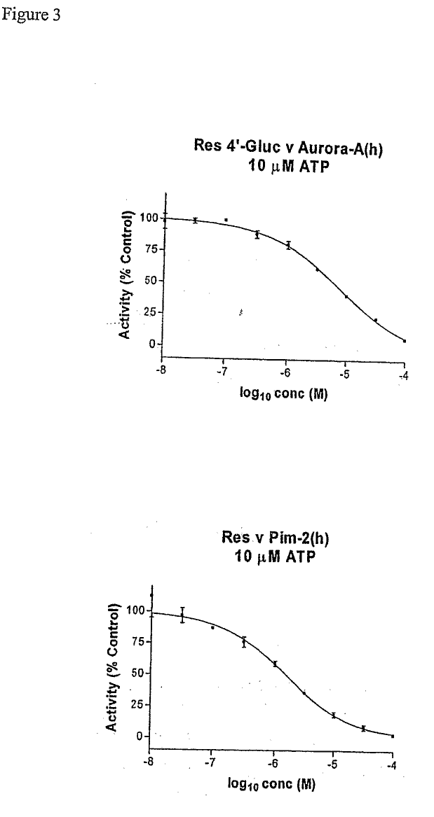

FIG. 4 shows graphs of the activity (as a percentage of the control) of human p70S6K in the presence of 10 or 100 μM ATP (panels A and B, respectively) and various concentrations of resveratrol.

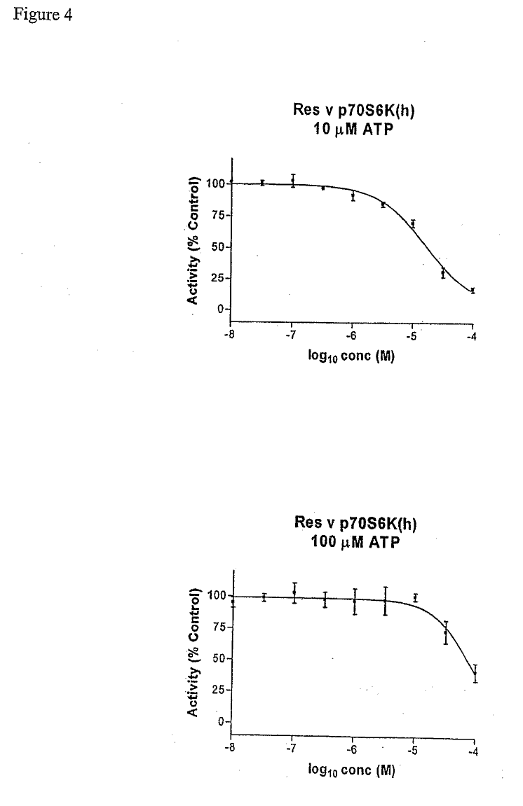

FIG. 5 shows graphs of the activity (as a percentage of the control) of human JAK3 (panel A) and NLK (panel B) in the presence of 10 μM ATP and various concentrations of resveratrol.

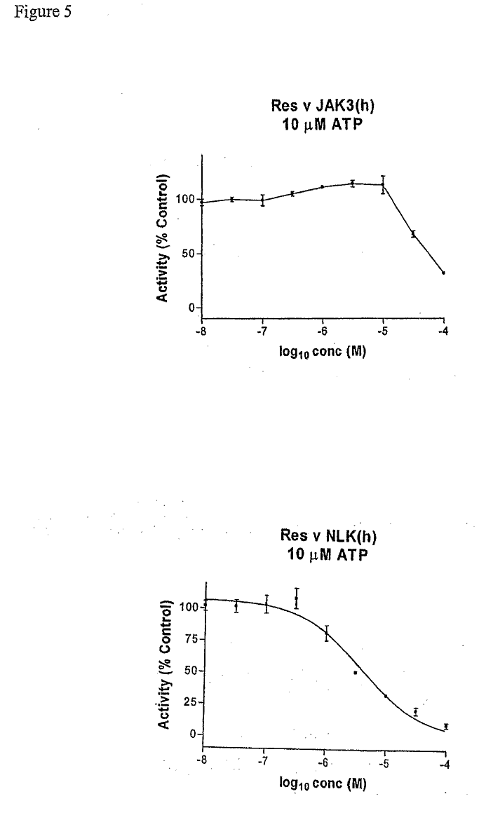

FIG. 6 is a graph showing dose dependent inhibition of the growth of hematopoietic cell line FL5.12 by resveratrol at 0.1, 1.0, 10, and 100 μM.

### DETAILED DESCRIPTION OF THE INVENTION

Provided herein are methods for treating and preventing diseases that benefit from the inhibition of a protein kinase, such as JAK2, Pim-1, Pim-2, S6K1, NLK and Rsk2. A method may comprise administering to a subject in need thereof a therapeutically effective amount of resveratrol or an analog or chemical derivative thereof. Exemplary diseases include cancers, neurodegenerative disorders and cardiac hypertrophy.

### Exemplary Compositions

Compounds for use herein include resveratrol (3, 5, 4′-trihydroxystilbene), its chemical derivatives, and structural neighbors as selective kinase inhibitors.

A compound may be a compound represented by formula 1:

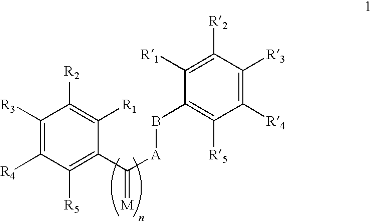

wherein, independently for each occurrence,

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, aralkyl, —SO₃H, monosaccharide, oligosaccharide, glycofuranosyl, glycopyranosyl, glucuronosyl, or glucuronide;

M represents O, NR, or S;

A—B represents a bivalent alkyl, alkenyl, alkynyl, amido, sulfonamido, diazo, ether, alkylamino, alkylsulfide, hydroxylamine, or hydrazine group; and

n is 0 or 1.

In a further embodiment, a compound may be a compound of formula 1 and the attendant definitions, wherein n is 0. In a further embodiment, a compound may be a compound of formula 1 and the attendant definitions, wherein n is 1. In a further embodiment, a compound may be a compound of formula 1 and the attendant definitions, wherein A—B is ethenyl. In a further embodiment, a compound may be a compound of formula 1 and the attendant definitions, wherein A—B is —CH₂CH(Me)CH(Me)CH₂—. In a further embodiment, a compound may be a compound of formula 1 and the attendant definitions, wherein M is O. In a further embodiment, a compound may be a compound of formula 1 and the attendant definitions, wherein R₁, R₂, R₃, R₄, R₅, R′₁, R′₂, R′₃, R′₄, and R′₅ are H. In a further embodiment, a compound may be a compound of formula 1 and the attendant definitions, wherein R₂, R₄, and R′₃ are OH. In a further embodiment, a compound may be a compound of formula 1 and the attendant definitions, wherein R₂, R₄, R′₂ and R′₃ are OH. In a further embodiment, a compound may be a compound of formula 1 and the attendant definitions, wherein R₃, R₅, R′₂ and R′₃ are OH. In a further embodiment, a compound may be a compound of formula 1 and the attendant definitions, wherein R₁, R₃, R₅, R′₂ and R′₃ are OH; In a further embodiment, a compound may be a compound of formula 1 and the attendant definitions, wherein R₂ and R′₂ are OH; R₄ is O—β—D-glucoside; and R′₃ is OCH₃. In a further embodiment, a compound may be a compound of formula 1 and the attendant definitions, wherein R₂ is OH; R₄ is O—β—D-glucoside; and R′₃ is OCH₃.

In a further embodiment, a compound may be a compound of formula 1 and the attendant definitions, wherein n is 0; A—B is ethenyl; and R₁, R₂, R₃, R₄, R₅, R′₁, R′₂, R′₃, R′₄, and R′₅ are H (trans stilbene). In a further embodiment, a compound may be a compound of formula 1 and the attendant definitions, wherein n is 1; A—B is ethenyl; M is O; and R₁, R₂, R₃, R₄, R₅, R′₁, R′₂, R′₃, R′₄, and R′₅ are H (chalcone). In a further embodiment, a compound may be a compound of formula 1 and the attendant definitions, wherein n is 0; A—B is ethenyl; R₂, R₄, and R′₃ are OH; and R₁, R₃, R₅, R′₁, R′₂, R′₄, and R′₅ are H (resveratrol). In a further embodiment, a compound may be a compound of formula 1 and the attendant definitions, wherein n is 0; A—B is ethenyl; R₂, R₄, R′₂ and R′₃ are OH; and R₁, R₃, R₅, R′₁, R′₄ and R′₅ are H (piceatannol). In a further embodiment, a compound may be a compound of formula 1 and the attendant definitions, wherein n is 1; A—B is ethenyl; M is O; R₃, R₅, R′₂ and R′₃ are OH; and R₁, R₂, R₄, R′₁, R′₄, and R′₅ are H (butein). In a further embodiment, a compound may be a compound of formula 1 and the attendant definitions, wherein n is 1; A—B is ethenyl; M is O; R₁, R₃, R₅, R′₂ and R′₃ are OH; and R₂, R₄, R′₁, R′₄, and R′₅ are H (3,4,2′,4′,6′-pentahydroxychalcone). In a further embodiment, a compound may be a compound of formula 1 and the attendant definitions, wherein n is 0; A—B is ethenyl; R₂ and R′₂ are OH, R₄ is O—β—D-glucoside, R′₃ is OCH₃; and R₁, R₃, R₅, R′₁, R′₄, and R′₅ are H (rhapontin). In a further embodiment, a compound may be a compound of formula 1 and the attendant definitions, wherein n is 0; A—B is ethenyl; R₂ is OH, R₄ is O—β—D-glucoside, R′₃ is OCH₃; and R₁, R₃, R₅, R′₁, R′₂, R′₄, and R′₅ are H (deoxyrhapontin). In a further embodiment, a compound may be a compound of formula 1 and the attendant definitions, wherein n is 0; A—B is —CH₂CH(Me)CH(Me)CH₂—; R₂, R₃, R′₂, and R′₃ are OH; and R₁, R₄, R₅, R′₁, R′₄, and R′₅ are H (NDGA).

A compound may also be a compound represented by formula 2:

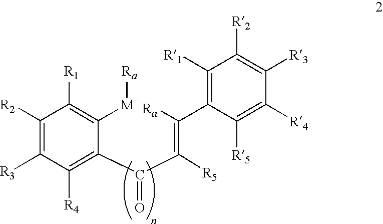

wherein, independently for each occurrence:

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, aryl, heteroaryl, aralkyl, —SO₃H, monosaccharide, oligosaccharide, glycofuranosyl, glycopyranosyl, glucuronosyl, or glucuronide; and

n is 0 or 1.

In a further embodiment, a compound is represented by formula 2 and the attendant definitions, wherein n is 0. In a further embodiment, a compound is represented by formula 2 and the attendant definitions, wherein n is 1. In a further embodiment, a compound is represented by formula 2 and the attendant definitions, wherein M is absent. In a further embodiment, a compound is represented by formula 2 and the attendant definitions, wherein M is O. In a further embodiment, a compound is represented by formula 2 and the attendant definitions, wherein Rₐ is H. In a further embodiment, the methods comprise an activating compound represented by formula 2 and the attendant definitions, wherein M is O and the two Rₐ form a bond.

In a further embodiment, a compound is represented by formula 2 and the attendant definitions, wherein R₅ is H. In a further embodiment, a compound is represented by formula 2 and the attendant definitions, wherein R₅ is OH. In a further embodiment, a compound is represented by formula 2 and the attendant definitions, wherein R₁, R₃, and R′₃ are OH. In a further embodiment, a compound is represented by formula 2 and the attendant definitions, wherein R₂, R₄, R′₂, and R′₃ are OH. In a further embodiment, a compound is represented by formula 2 and the attendant definitions, wherein R₂, R′₂, and R′₃ are OH. In a further embodiment, a compound is represented by formula 2 and the attendant definitions, wherein R₂ and R₄ are OH. In a further embodiment, a compound is represented by formula 2 and the attendant definitions, wherein R₂, R₄, R₅, R′₃, and R′₄ are OH.

In a further embodiment, a compound is represented by formula 2 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, a compound is represented by formula 2 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a compound is represented by formula 2 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H. In a further embodiment, a compound is represented by formula 2 and the attendant definitions, wherein n is 1; M is O; the two Rₐ form a bond; R₂, R₄, R₅, R′₃, and R′₄ are OH; and R₁, R₃, R′₁, R′₂, and R′₅ are H (quercetin).

In certain embodiments, a compound has a structure represented by formula 1 or 2, with the proviso that the compound is not a specific compound, e.g., resveratrol or piceatannol.

Also included are pharmaceutically acceptable addition salts and complexes of the compounds of formulas 1 and 2. In cases wherein the compounds may have one or more chiral centers, unless specified, the compounds contemplated herein may be a single stereoisomer or racemic mixtures of stereoisomers.

In cases in which the compounds have unsaturated carbon-carbon double bonds, both the cis (Z) and trans (B) isomers are contemplated herein. In cases wherein the compounds may exist in tautomeric forms, such as keto-enol tautomers, such as

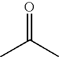

and

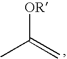

each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the compounds of formulas 1 and 2. Prodrugs are considered to be any covalently bonded carriers that release the active parent drug in vivo. Metabolites, such as in vivo degradation products, of the compounds described herein are also included.

Analogs and derivatives of the above-described compounds can also be used. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Resveratrol may be obtained commercially, e.g., from Sigma.

Additional compounds are those described, e.g., in U.S. published applications numbers 2005/0096256, 2005/0096256, and PCT applications publication numbers WO 05/002672 and WO 05/002555, all of which are specifically incorporated by reference herein.

In one embodiment, quercetin, an exemplary xenohormetic compound behaves similarly to resveratrol in its ability to inhibit kinases including S6K, PKC, and AMPK (AMP kinase) and inhibition of signaling through a Jak pathway (Davies, S. P. et al., Biochem J. 351:95-105 (2000); Muthian, G. and Bright, J. J., J. Clin. Immunol. 24:542-52 (2004).

### Exemplary Diseases

Provided herein are methods for treating or preventing a disease or condition that may benefit from inhibiting a kinase, such as JAK2, Pim-1, Pim-2, NLK, S6K and Rsk2. A method may comprise administering to a subject, e.g., a subject in need thereof, a therapeutically effective amount of a compound described herein. The subject can be a human, a non-human primate, a bovine, an ovine, an equine, a porcine, a sheep, a canine, a feline or a rodent (mouse or rat).

A subject in need of treatment may be a subject who has been diagnosed with a disease that is treatable as described herein. A subject in need of prevention of treatment may be a subject who is likely to develop the disease due to, e.g., hereditary reasons.

Exemplary diseases and conditions that may be treated include those in which a kinase, e.g., JAK2, Pim-1, Pim-2, NLK, S6K and Rsk2, is overexpressed or upregulated. A disease may be a hyper-proliferative disease, e.g., a malignant (cancer) or benign growth. Exemplary cancers include those of the hematopoietic system, such as myeloproliferative disorders: chronic myeloproliferative disorders; atypical myeloproliferative disorders, e.g., chronic neutrophilic leukemia (CNL); polycythemia vera, essential thrombocythemia, myeloid metaplasia with myelofribrosis. Other lymphomas and leukemias that can be treated as described herein include lymphoid tumors, diffuse B cell lymphoma, chronic lymphocytic leukemia, non-Hodgkin's lymphoma, FLT3-mediated acute myelogenous leukemia, chronic myelomonocytic leukemia, acute myeloid leukemia, Philadelphia-chromosome (Ph)-negative chronic myelogenous leukemia (CML), megakaryocytic acute myelogenous leukemia, and those characterized by myelodysplastic syndromes.

Pim family kinases enhance tumor growth of prostate cancer cells (Chen et al. (2005) Mol Cancer Res. 3(8):443). Accordingly, the compounds described herein may be used for treating prostate cancer. Since Pim-1 and -2 are also involved in hematopoietic tumorigenesis, the compounds described herein may also be used for treating such diseases.

S6K is overexpressed in breast adenocarcinomas (Filonenko et al. (2004) Exp. Oncol. 26:294). Accordingly, the compounds described herein may be used for treating breast adenocarcinomas.

Pim-2 is involved in promoting the growth and survival of nontransformed hematopoietic cells (8). Accordingly, the compounds described herein may also be used to inhibit the growth and survival of nontransformed hematopoietic cells.

Pim-1 and Pim-2 are essential components of an endogenous pathway that regulates T cell growth and survival. In addition, Pim kinase deficiency enhances rapamycin action in vivo (Fox et al. (2005) J. Exp. Med. 201:259). Accordingly, the compounds described herein may be used as immunosuppressant compounds, which may be administered together with rapamycin or other immunosuppressant compounds to increase the effect of rapamycin or the other immunosuppressant compound. Exemplary conditions in which immunosuppression maybe useful include transplant rejections, in which the immunosuppressant drug delays or prevents transplant rejection. Graft versus host disease may be prevented by treating the graft with a compound described herein.

Autoimmune and immune related disorders and diseases may also be treated or prevented as described herein. Exemplary autoimmune diseases and immune related disorder include systemic lupus erythematosis, rheumatoid arthritis, osteoarthritis, juvenile chronic arthritis, a spondyloarthropathy, systemic sclerosis, an idiopathic inflammatory myopathy, Sjogren's syndrome, systemic vasculitis, sarcoidosis, autoimmune hemolytic anemia, autoimmune thrombocytopenia, thyroiditis, diabetes mellitus, immune-mediated renal disease, a demyelinating disease of the central or peripheral nervous system, idiopathic demyelinating polyneuropathy, Guillain-Barr syndrome, a chronic inflammatory demyelinating polyneuropathy, a hepatobiliary disease, infectious or autoimmune chronic active hepatitis, primary biliary cirrhosis, granulomatous hepatitis, sclerosing cholangitis, inflammatory bowel disease, gluten-sensitive enteropathy, Whipple's disease, an autoimmune or immune-mediated skin disease, a bullous skin disease, erythema multiforme, contact dermatitis, psoriasis, an allergic disease, asthma, allergic rhinitis, atopic dermatitis, food hypersensitivity, urticaria, an immunologic disease of the lung, eosinophilic pneumonias, idiopathic pulmonary fibrosis, hypersensitivity pneumonitis, systemic lupus erythematosus, scleroderma, and arthritis.

Pim activity has also been shown to be important in the pathogenesis of vascular smooth muscle proliferation in vessel injury models (Katakmi et al. (2004) J. Biol. Chem. 279:54743). Since smooth muscle cell proliferation is a key feature of neointima formation, atherosclerosis, restenosis, graft failure, angiogenesis and/or solid tumour growth, all of these conditions may be treated or prevented in a subject by administration to the subject of a compound described herein.

Neurodegenerative diseases that may be treated include neurodegenerative diseases, e.g., Alzheimer's, Amyotrophic Lateral Sclerosis (ALS), and Parkinson's disease. Another class of neurodegenerative diseases includes diseases caused at least in part by aggregation of poly-glutamine. Diseases of this class include: Huntington's Diseases, Spinalbulbar Muscular Atrophy (SBMA or Kennedy's Disease) Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCA7), and Spinocerebellar Ataxia 12 (SCA12).

Additional diseases that may benefit from inhibiting the activity of a kinase include cardiac hypertrophy and other cardiovascular diseases, such as angina pectoris, arrhythmias, myocardial infarction, cardiac hypertrophy, hypotension, hypertension, e.g. essential hypertension, renal hypertension, or pulmonary hypertension, thrombosis, arteriosclerosis, cerebral vasospasm, subarachnoid hemorrhage, cerebral ischemia, cerebral infarction, peripheral vascular disease, Raynaud's disease, and kidney disease, e.g. renal failure.

The compounds described herein can be in any pharmaceutically acceptable salt form, i.e., relatively non-toxic, organic or inorganic salts, e.g., hydrochloride salts, sulfate salts, bisulfate salts, borate salts, nitrate salts, acetate salts, phosphate salts, hydrobromide salts, laurylsulfonate salts, glucoheptonate salts, oxalate salts, oleate salts, laurate salts, stearate salts, palmitate salts, valerate salts, benzoate salts, naphthylate salts, mesylate salts, tosylate salts, citrate salts, lactate salts, maleate salts, succinate salts, tartrate salts, fumarate salts, and the like. See, e.g., Berge, et al., J. Pharm. Sci., 66:1-19 (1977).

Compounds can be delivered locally, e.g., to a tissue or organ within a subject, such as by injection. An injection or infusion may be intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal. Routes of topical administration include skin, nasal, bucal, mucosal, rectal, or vaginal applications. Administration may also be systemic, peripheral, enteral or parenteral. Subcutaneous implantation for sustained release of the compounds may also be a suitable route of administration.

The compounds may be formulated for transdermal delivery systems, e.g., patches, gels, tapes and creams; transmucosal delivery systems, e.g., patches, tablets, suppositories, pessaries, gels and creams; oral delivery systems, e.g., tablets and capsules; topical delivery systems, e.g., gels and solutions; injectable drag delivery systems, e.g., solutions, suspensions, gels, microspheres and polymeric injectables.

A compound may be administered to a subject at a dose of about 0.01 to 10 mg/kg or 0.1 to 1 mg/kg. A compound may be administered daily, weekly, biweekly, every two weeks, every four weeks, monthly or every few months.

Administration of a compound to a subject may be followed by measuring a factor in the subject, e.g., the level of the compound in a tissue or bodily fluid, such as blood, serum or urine. The level of protein or activity of one or more kinases may also be determined. A lower level of activity of one or more kinases in tumor cells may indicate that the treatment is at least partially successful.

Compounds described herein could also be taken as one component of a multi-drug complex or as a supplement in addition to a multi-drug regimen, e.g., in combination with a chemotherapeutic agent. The compounds may be administered simultaneously or successively. For example, a composition comprising one or more compounds described herein and one or more chemotherapeutic agents may be administered to a subject. Compositions comprising two or more compounds described herein alone or together with a chemotherapeutic agent may also be administered.

Chemotherapeutic agents that may be coadministered with compounds described herein as having anti-cancer activity (e.g., compounds that induce apoptosis, compounds that reduce lifespan or compounds that render cells sensitive to stress) include: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, fligrastim, fludarabine, fludrocortisone, fluorouracil, fluoxymesterone, flutamide, gemcitabine, genistein, goserelin, hydroxyurea, idarubicin, ifosfamide, imatimib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, ratitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; chromatin disruptors.

The methods may be advantageous over combination therapies known in the art because it allows conventional chemotherapeutic agents to exert greater effect at lower dosage. In a preferred embodiment, the effective dose (ED₅₀) for a chemotherapeutic agent or combination of conventional chemotherapeutic agents when used in combination with a compound described herein is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a compound described herein can be at least 2 fold greater than the TI for conventional chemotherapeutic regimen alone, and even more preferably at 5 fold, 10 fold or even 25 fold greater.

### Methods for Predicting the Effectiveness of a Treatment

Also provided herein are methods for predicting whether a subject is likely to respond to therapy with resveratrol or other compound described herein. The methods are based at least in part on the observation that enhanced Pim activity could be detected in tumor cells before the initiation of treatment using a diagnostic test. This type of personalized medicine could enable doctors to prescribe a treatment with a compound described herein or the addition of a compound described herein to a current treatment in the event that their tumor profile includes the overexpression or enhanced activity of a kinase selected from the group consisting of JAK2, Pim-1, Pim-2, S6K, NLK or Rsk2.

An exemplary method comprises determining the level of protein or activity of a protein kinase, e.g., JAK2, Pim-1, Pim-2, S6K, NLK or Rsk2 in a tissue of a subject, wherein the presence of an elevated level of protein or activity relative to a normal control indicates that the subject is likely to respond positively to administration of a compound described herein. A different level of protein or activity may be a difference of at least about 50%, 75%, 100% (2 fold), 3 fold, 5 fold, or 10 fold or more. A normal control may be the level of protein or activity of the kinase in a tissue of similar origin to that of the tissue obtained from the subject.

A method may comprise one or more of the following steps: (i) obtaining a tissue sample, e.g., a tumor tissue sample, from a subject; (ii) determining the level of protein or activity of a kinase in the tissue sample; and, if the level is higher than in a control tissue, (iii) administering to the subject a therapeutically effective amount of a compound described herein.

Determining the level of protein in a tissue sample can be done, e.g., with an immunoassay, such as an assay that utilizes biotin and avidin or streptavidin, ELISAs, RIAs, Western blots, and immunoprecipitation. Diagnostic methods may use an antibody that specifically binds to a kinase. Other diagnostic assays may comprise the use of nucleic acids, e.g., for determining the level of RNA, such as mRNA, that is predictable of the level of protein. The agent that is used in a diagnostic assay, e.g., an antibody or a nucleic acid, may be labeled and/or linked, covalently or not, to a solid surface.

Determining the level of activity of a kinase in a tissue may be conducted by contacting the tissue or a cell extract thereof or purified kinase therefrom, with a substrate, e.g., a peptide substrate, and any other necessary component, e.g., ATP, and determining the level of phosphorylation of the substrate.

A description of the kinases discussed herein is set forth below.

JAK2 (Janus kinase 2; GeneID: 3717) is a protein tyrosine kinase that is involved in a specific subset of cytokine receptor signaling pathways. The nucleotide and amino acid sequences of human JAK2 are set forth in GenBank Accession numbers NM_{—}004972 and NP_{—}004963, respectively.

Pim-1 (GeneID: 5292), also referred to as oncogene PIM1; pim-1 kinase 44 kDa isoform; and pim-1 oncogene (proviral integration site 1), encodes a protein kinase that is upregulated in prostate cancer. The nucleotide and amino acid sequences of human Pim 1 are set forth in GenBank Accession numbers NM_{—}002648 and NP_{—}002639, respectively.

Pim-2 (GeneID: 11040) is also referred to as PIM2 oncogene. The nucleotide and amino acid sequences of human Pim 2 are set forth in GenBank Accession numbers NM_{—}006875 and NP_{—}006866, respectively.

S6K (GeneID: 6198) is also referred to as RPS6KB1 ribosomal protein S6 kinase 70 kDa polypepide 1; PS6K; S6K1; STK14A; p70-S6K; p70-alpha; and p70(S6K)-alpha, is a member of the RSK (ribosomal S6 kinase) family of serine/threonine kinases. This kinase contains 2 non-identical kinase catalytic domains and phosphorylates several residues of the S6 ribosomal protein. The kinase activity of this protein leads to an increase in protein synthesis and cell proliferation. The nucleotide and amino acid sequences of human S6K are set forth in GenBank Accession numbers NM_{—}003161 and NP_{—}003152, respectively.

NLK (GeneID: 51701) is also referred to as nemo like kinase. The nucleotide and amino acid sequences of human NLK are set forth in GenBank Accession numbers NM_{—}016231 and NP_{—}057315, respectively.

Rsk2 (GeneID: 6197) is also referred to as ribosomal protein S6 kinase, 90 kDa, polypeptide 3; RPS6KA3; RSK; HU-3; RSK2; MRX19; ISPK-1; p90-RSK3; pp90RSK2; MAPKAPK1B; and S6K-alpha3. This gene encodes a member of the RSK (ribosomal S6 kinase) family of serine/threonine kinases. This kinase contains 2 non-identical kinase catalytic domains and phosphorylates various substrates, including members of the mitogen-activated kinase (MAPK) signalling pathway. The activity of this protein has been implicated in controlling cell growth and differentiation. Mutations in this gene have been associated with Coffin-Lowry syndrome (CLS). The nucleotide and amino acid sequences of human Rsk2 are set forth in GenBank Accession numbers NM_{—}004586 and NP_{—}004577, respectively.

The present description is further illustrated by the following examples, which should not be construed as limiting in any way. The contents of all cited references (including literature references, issued patents, published patent applications and GenBank Accession numbers as cited throughout this application) are hereby expressly incorporated by reference. When definitions of terms in documents that are incorporated by reference herein conflict with those used herein, the definitions used herein govern.

### EXAMPLES

### Example 1

### Resveratrol Inhibits Certain Kinases

We have screened resveratrol against a panel of 100 kinases and discovered a striking specificity as compared to known kinase inhibitors. Kinase reactions were performed in the presence of 10 or 100 μM ATP and with concentrations of resveratrol of 0.01 μM, 0.03 μM, 0.1 μM, 0.3 μM, 1 μM, 3 μM, 10 μM, 30 μM and 100 μM.

The results are shown in the attached figures. The results show in particular that resveratrol is a specific inhibitor of JAK2, Pim-1, Pim-2, p70S6K, NLK and Rsk2. Rsk2 was inhibited about 68% with 20 μM resveratrol.

The data generated indicates that resveratrol is a kinase inhibitor with more selectivity than previously thought. Most of the kinases in the screen, including the previously identified resveratrol targets Src and PKC (1), are either not inhibited or only weakly inhibited by resveratrol at a concentration of 20 micromolar. This concentration is well above the serum levels expected to be achievable in animals following oral or intravenous administration (2).

Interestingly, the best targets found in our screen are kinases believed to play a role in various human pathologies including cancers, neurodegenerative disorders, cardiac hypertrophy, and other diseases. Most importantly, JAK2 and Pim-1/2 are key players in a signaling cascade that is critical to the development of many leukemias and lymphomas (3). Other disease-related targets include NLK, S6K1, and Rsk2. The IC50s of resveratrol for all of these kinases are significantly lower than those of the best currently reported targets.

Based on our results, resveratrol may be useful for inhibiting the Pim gene family (responsible for more than 50% of the transforming events in leukemias and lymphomas) as well as the upstream kinase JAK2 in hematopoietic cancers. Current research suggests that this may have a profound impact on the survival and growth of tumor cells. Due to its role in regulating lymphoid survival during the immune response, Pim inhibitors like resveratrol may also be useful in the treatment of autoimmune diseases.

There is also some recent evidence that Pims may play a bigger role than previously suspected outside of the hematopoetic system, including upregulation in prostate cancer. In the future it may become apparent that Pims play a more global role in tumorigenesis and thus provide more therapeutic applications for resveratrol.

The inhibition of the Pim gene family, which can contribute to the transforming events in human leukemias and lymphomas (7), is especially exciting, as the IC50's are 871 nM and 1490 nM for Pim-1 and Pim-2 respectively. At this point resveratrol could be considered the most selective Pim family inhibitor, as LY294002, the only known Pim inhibitor (3), is quite promiscuous (and in fact, is marketed as a PI3K inhibitor). Resveratrol, on the other hand, displays no inhibition of PI3K or most other kinases at concentrations up to 20 micromolar. Pim overexpression has been shown in diffuse B cell lymphoma (3), chronic lymphocytic leukemia (4), non-Hodgkin lymphoma (4,5) and prostate cancer, and FLT3 -mediated acute myelogenous leukemia (3). Pim has also recently been shown to be involved in non-pathologic expansion during the immune response (6,7, 8). Knowing the importance of Pim as a transforming event in human cancers of the hematopoietic system, resveratrol, its derivatives, and structural neighbors may be useful as inhibitors of Pims in the treatment of lymphoid tumors. Additionally, resveratrol's effects on JAK2/STAT3 signalling through inhibition of JAK2, at an IC50 of 1470 nM, could provide cooperative or possibly synergistic effects on proliferation and apoptosis in tumor cells especially in the hematopoietic system (3).

### Example 2

### Dose Dependent Inhibition of the Growth of Hematopoietic Cell Line FL5.12 by Resveratrol.

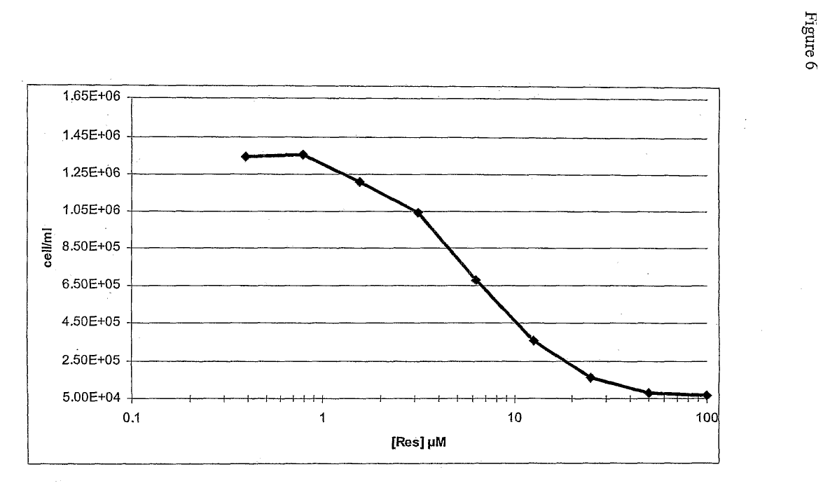

Hematopoietic cell line FL5.12 cells were seed at equal density and grown in the presence of IL-3, a growth factor. Cells were then treated for 24 hours with 0.1, 1, 10 and 100 μM resveratrol. Cells were counted with a Coulter particle counter to determine cell inhibition by resveratrol. FIG. 6 is a graph showing dose dependent inhibition of FL5.12 cell growth by resveratrol.

### REFERENCES

1) Yu, R., V. Hebbar, et al. Resveratrol inhibits phorbol ester and UV-induced activator protein 1 activation by interfering with mitogen-activated protein kinase pathways. 2001 Mol Pharmacol 60(1): 217-24.
2) Goldberg D M, Yan J, Soleas G J. Absorption of three wine-related polyphenols in three different matrices by healthy subjects. Clin Biochem. 2003 February; 36(l):79-87.
3) Ravi Amaravadi and Craig B. Thompson. The survival kinases Akt and Pim as potential pharmacological targets. J Clin Invest. 2005 October; 115(10):2618-24.
4) Cohen A M et al. Increased expression of the hPim-2 gene in human chronic lymphocytic leukemia and non-Hodgkin lymphoma.

### Leuk Lymphoma. 2004 May; 45(5):951-5.

5) Libra M, Capello D, Gloghini A, Laura P, Berra E, Cerri M, Gasparotto D et al. Analysis of aberrant somatic hypermutation (SHM) in non-Hodgkin's lymphomas of patients with chronic HCV infection. J Pathol. 2005 May; 206(l):87-91.
6) Mikkers H et al. Mice deficient for all PIM kinases display reduced body size and impaired responses to hematopoietic growth factors.

### Mol Cell Biol. 2004 July; 24(13):6104-15.

7) Fox C J, Hammerman P S, Thompson C B. The Pim kinases control rapamycin-resistant T cell survival and activation.

### J Exp Med. 2005 Jan. 17; 201(2):259-66.

8) Hammerman P S, Fox C J, Birnbaum M T, Thompson C B. Pim and Akt oncogenes are independent regulators of hematopoietic cell growth and survival.

### Blood. 2005 Jun. 1; 105(11):4477-83.

### Incorporation by Reference

The contents of all cited references (including literature references, issued patents, published patent applications and Genbank Accession numbers as cited throughout this application) are hereby expressly incorporated by reference.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for treating or preventing a disease associated with excessive JAK2, Pim-1, Pim-2, S6K, NLK or Rsk2 activity or protein level in a subject, comprising administering to a subject in need thereof a therapeutically effective amount of a compound represented by formula 1 or 2 or a salt thereof.

2. The method of claim 1, wherein the disease is selected from the group consisting of diffuse B cell lymphoma, chronic lymphocytic leukemia, non-Hodgkin's lymphoma, prostate cancer, and FLT3-mediated acute myelogenous leukemia.

3. The method of claim 1, wherein the disease is cardiac hypertrophy.

4. A method for increasing the potency of an immunosuppressant, comprising adding to a composition comprising an immunosuppressant a compound represented by formula 1 or 2 or a salt thereof.

5. A method for reducing an immune reaction in a subject, comprising administering to a subject a compound represented by formula 1 or 2 or a salt thereof.

6. The method of claim 5, further comprising administering an immunosuppressant to the subject.

7. The method of claim 6, wherein the immunosuppressant is rapamycin.

8. The method of claim 5, for preventing graft versus host disease or an autoimmune disease.

9. A method for determining whether a disease can be treated or prevented with a compound represented by formula 1 or 2 or a salt thereof in a subject, comprising determining the level of protein or activity of JAK2, Pim-1, Pim-2, S6K, NLK or Rsk2 in a cell of the subject, wherein a higher level of protein or activity in the cell of the subject relative to a control indicates that the disease can be treated or prevented in the subject by administration of a compound represented by formula 1 or 2 or a salt thereof.

10. A method for treating or preventing a disease associated with excessive JAK2, Pim-1, Pim-2, S6K, NLK or Rsk2 activity or protein level in a subject, comprising (i) determining whether the disease can be treated or prevented with a compound represented by formula 1 or 2 or a salt thereof, as set forth in claim 9; and if the result is positive, (ii) administering to the subject a therapeutically effective amount of a compound represented by formula 1 or 2 or a salt thereof.

11. The method of claim 1, further comprising determining the level of activity of a kinase selected from the group consisting of JAK2, Pim-1, Pim-2, S6K, NLK or Rsk2 in a cell of the subject.

12. The method of claim 1, further comprising determining the level of the compound in a bodily fluid of the subject.
